(19) United States
(12) Patent Application Publication
(10) Pub. No.: US 2018/0133059 A1
(43) Pub. Date: May 17, 2018

(54) EYE-MASKS CONFIGURED TO INTEGRATE WITH HEADPHONES AND OTHER EXTERNAL SYSTEMS

(71) Applicant: Muzik, LLC, Raleigh, NC (US)

(72) Inventor: Jason Hardi, Raleigh, NC (US)

(21) Appl. No.: 15/810,443

(22) Filed: Nov. 13, 2017

Related U.S. Application Data
(60) Provisional application No. 62/420,928, filed on Nov. 11, 2016.

(51) Int. Cl. A61F 9/04; H04R 1/10

(52) U.S. Cl. CPC A61F 9/04; H04R 1/1008; H04R 2420/07; H04R 1/1091; A61F 2250/0002; H04R 1/1058

(57) ABSTRACT

An eye-mask can include an exterior surface that is configured to face away from a wearer and an interior surface, that is opposite the exterior surface, where the interior surface can be configured to contact the wearer and cover eyes of the wearer during use. A first removable coupling, coupled to a first end of the exterior surface, the first removable coupling configured to removably couple to a first ear piece of a pair of headphones. A second removable coupling, coupled to a second end of the exterior surface opposite the first end, the second removable coupling configured to removably couple to a second ear piece of the pair of headphones.

### CROSS-REFERENCE TO RELATED APPLICATION

This application claims priority to U.S. Provisional Patent Application No. 62/420,928, entitled EYE-MASK INTEGRATED WITH HEADPHONES, filed on Nov. 11, 2016 in the United States Patent and Trademark Office, the entirety of which is incorporated herein by reference.

### FIELD

The present inventive concept relates to the field of electronics in general, and more particularly, to headphones.

### BACKGROUND

It is known to provide audio headphones with wireless connectivity which can support streaming of audio content to the headphones from a mobile device, such as a smartphone. In such approaches, audio content that is stored on the mobile device is wirelessly streamed to the headphones for listening. Further, such headphones can wirelessly transmit commands to the mobile device for controlled streaming. For example, the audio headphones may transmit commands such as pause, play, skip, etc. to the mobile device which may be utilized by an application executed on the mobile device. Accordingly, such audio headphones support wirelessly receiving audio content for playback to the user as well as wireless transmission of commands to the mobile device for control of the audio playback to the user on the headphones.

### SUMMARY

Embodiments according to the present inventive concept can provide an eye-mask configured to integrate with headphones. Pursuant to these embodiments, an eye-mask can include an exterior surface that is configured to face away from a wearer and an interior surface, that is opposite the exterior surface, where the interior surface can be configured to contact the wearer and cover eyes of the wearer during use. A first removable coupling, coupled to a first end of the exterior surface, the first removable coupling configured to removably couple to a first ear piece of a pair of headphones. A second removable coupling, coupled to a second end of the exterior surface opposite the first end, the second removable coupling configured to removably couple to a second ear piece of the pair of headphones.

In some embodiments, an eye-mask can include an exterior surface configured to face away from a wearer and an interior surface, opposite the exterior surface, where the interior surface is configured to contact the wearer and cover eyes of the wearer during use. First and second removable couplings can be coupled to first and second ends of the exterior surface, where the first and second removable couplings can be configured to integrate the eye-mask with a pair of headphones.

In some embodiments, an eye-mask can include an exterior surface configured to face away from a wearer and an interior surface, opposite the exterior surface, where the interior surface can be configured to contact the wearer and cover eyes of the wearer during use. A first removable coupling can be coupled to a first end of the exterior surface, where the first removable coupling can include a first electrical connector configured to removably couple to a first ear piece electrical connector of a pair of headphones and a second removable coupling can be coupled to a second end of the exterior surface, the second removable coupling comprising a connector configured to removably couple to a second ear piece connector of the pair of headphones.

### BRIEF DESCRIPTION OF THE FIGURES

FIG. 1 is an illustration of an eye-mask integrated with headphones in some embodiments according to the inventive concept.

FIG. 2 is an illustration of an eye-mask configured to integrate with headphones in some embodiments according to the inventive concept.

FIGS. 3A-3C is an illustration of headphones configured to integrate with an eye-mask in some embodiments according to the inventive concept.

FIG. 4 is an illustration of an exterior of an eye-mask configured to integrated with headphones in some embodiments according to the inventive concept.

FIG. 5 is an illustration of an interior of an eye-mask configured to integrated with headphones in some embodiments according to the inventive concept.

FIG. 6 is a block diagram illustrating an architecture of the headphones configured with an eye-mask in some embodiments according to the inventive concept.

FIG. 7 is a block diagram illustrating an architecture of a cross-platform application programming interface for headphones integrated with an eye-mask in some embodiments according to the inventive concept.

### DESCRIPTION OF EMBODIMENTS ACCORDING TO THE INVENTIVE CONCEPT

Advantages and features of the present disclosure and methods of accomplishing the same may be understood more readily by reference to the following detailed description of preferred embodiments and the accompanying drawings. The present disclosure may, however, be embodied in many different forms and should not be construed as being limited to the embodiments set forth herein. Rather, these embodiments are provided so that this disclosure will be thorough and complete and will fully convey the concept of the present disclosure to those skilled in the art, and the present disclosure will only be defined by the appended claims. Like reference numerals refer to like elements throughout the specification.

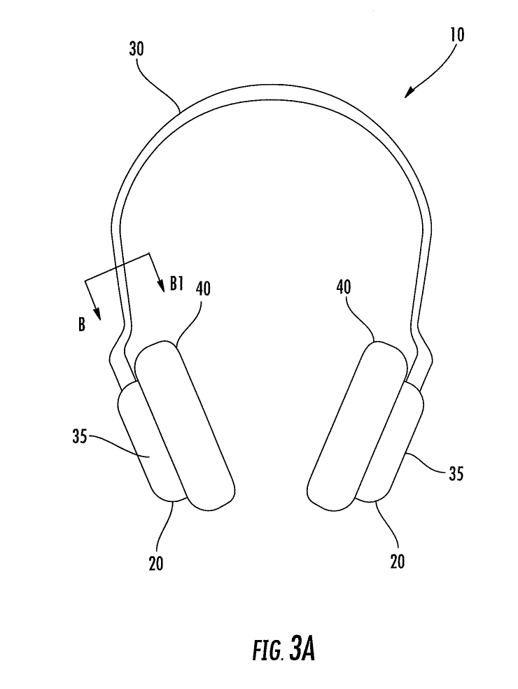

It will be understood that the headphones described herein (such as those shown in FIG. 3 herein) can be the headphones described in U.S. patent application Ser. No. 15/628,206, entitled Audio/Video Wearable Computer System with Integrated Projector, filed on Jun. 20, 2017, in the United States Patent and Trademark Office, the entirety of which is incorporated herein by reference. In particular, the headphones described herein can include the same features and functionality as those described in the above referenced application which, when integrated with the eye-mask in some embodiments according to the inventive concept, can provide control over the eye-mask and can receive input from the eye-mask.

In some embodiments according to the invention, what is sometimes referred to as an “eye-mask” or “sleep mask” can be integrated with headphones which may provide a convenient configuration for wearers. It will be understood that the term “integrated” as used herein can include the eye-mask being mechanically integrated with the headphones and/or functionally integrated with the headphones. Mechanical integration with the headphones may obviate the need for a separate strap for the eye-mask. For example, in some embodiments according to the invention the eye-mask can be removably coupled to the headphones by loops which wrap around the ear pieces of the headphones so that the eye-mask may be worn without a separate attachment to the wearer (such as a strap). Accordingly, in some embodiments according to the invention, the opposite ends of the eye-mask may be separated from one another and each of the respective ends can include a loop which is configured to be passed over a respective one of the ear pieces to secure the eye-mask to the headphones.

Functional integration with the headphones may provide communications and control of operations supported by the eye-mask. For example, in some embodiments according to the invention, the integration with the headphones can provide support for functions that are supported by the eye-mask such as a signal light which may be used to call a flight attendant or indicate that assistance is desired. In still further embodiments according to the invention, the eye-mask can include an internal light that operates under control of the headphones. The internal light may be visible to the wearer and may be used for a “wake-up” function that gradually introduces light to the interior of the mask so that the user is gradually awakened without having to remove the mask. In still further embodiments, the internal light may operate under control of an external system (via the headphones) such as when a flight attendant wishes to “wake” passengers at a particular time. The functional integration can be provided via a physical connection (e.g., a connector) or a wireless connection (e.g., a Bluetooth connection).

In still further embodiments, “integration” can provide both mechanical and functional integration of the headphones with the eye-mask. For example, in some embodiments according to the invention, the eye-mask can be removably coupled to the headphones using, for example, a connector or other interface that provides a mechanical and functional interface to the headphones.

In still further embodiments according to the invention, certain functions supported by the eye-mask can be activated when the eye-mask is coupled to the headphones. For example, when the eye-mask is detected as being integrated with the headphones, the headphones may automatically mute any incoming audio and/or video signals so as not to disturb the wearer. Still further, an auto wake-up mode may be enabled to wake the wearer after a selected time interval whereupon a tone may be introduced via the headphones or light may be introduced as described above. In some embodiments, when the eye-mask is detected as being integrated with the headphones, the headphones may select predetermined music selections for audio. For example, the audio content selected may be pre-selected by the wearer for sleep, may have particular “sleep timer” after which audio is muted, a preset volume level and tone settings, and the like. In some embodiments, the camera(s) can be enabled or disabled when the eye-mask is detected as being integrated with the headphones. In some embodiments, the projector included with the headphones can be enabled or disabled when the eye-mask is detected as being integrated with the headphones. In some embodiments, the headphones may update the wearers status on social media (such as to “DND”). In some embodiments, the inputs on the headphones may be assigned with predetermined functions when the eye-mask is detected as being integrated with the headphones.

In still further embodiments according to the invention, an exterior surface of the eye-mask may include a display which operates under the control of the headphones or other system. Accordingly, images on the display may not be visible to the wearer but may be visible to outside observers. Such a display may be used for, for example, a message to outside observers, advertising, etc.

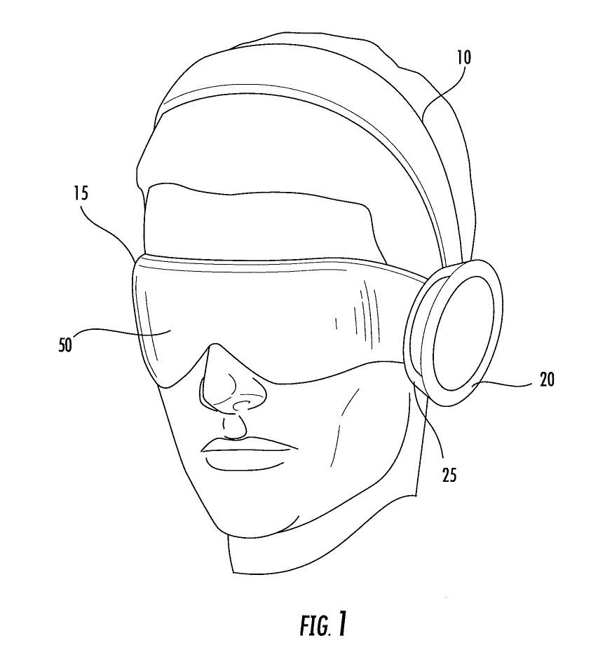

FIG. 1 is a perspective view of an eye-mask 15 removably coupled to headphones 10 in some embodiments. The headphones 10 may include first and second ear pieces 20 located on opposite ends of the headphones 10 and configured to be placed on the ears of a user. As further shown in FIG. 1, the eye-mask 15 is configured to cover the eyes of the wearer and be removeably coupled to the ear pieces 20 by, for example, loops 25. According to FIG. 1, in some embodiments the loops 25 can be elastic so that they can be stretched around the ear pieces 20 and provide tension onto the eye-mask 15 so that it remains securely in place over the wearer's eyes during use. It will be understood that the function of the loops 25 can be provided by any mechanism that provides removable coupling to the headphones 10 so that the eye-mask 15 is integrated with the headphones 10 during use.

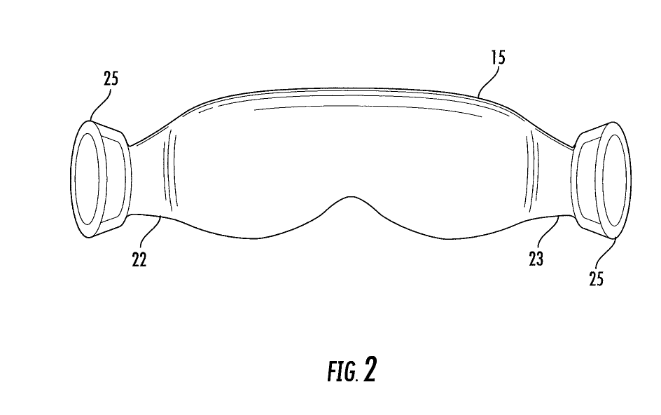

FIG. 2 is a schematic representation of the eye-mask 15 including the loops 25 shown on opposing ends of the eye-mask 15 each of which can be removably coupled to a respective one of the ear pieces 20 of the headphones 10. As shown in FIG. 2, the loops 25 are separated from one another so that the eye-mask 15 need not include a strap which couples a first end of the eye-mask 22 to a second end of the eye-mask 23. As described above, the loops 25 can be replaced with any mechanism that provides removably coupling the eye-mask 15 to the headphones 10.

FIG. 3A is a schematic illustration of the headphones 10 including the left and right ear pieces 20 connected by a headband portion 30. Each of the ear pieces 20 includes a respective housing 35 and cushion 40. As shown in FIG. 3A, each of the housings 35 includes an intermediate portion between the respective cushion 40 and the headband 30 that can accommodate the loops 25 shown in FIGS. 1 and 2. In operation, the loops 25 can be stretched over the cushions 40 to rest against the intermediate portion of the housing 35 to provide tension onto the eye-mask 15 so that it is secured to the wearer as shown in FIG. 3B.

As shown in FIG. 3C, in some embodiments according to the invention, the left and right end portions 22 and 23 of the eye-mask 15 can be coupled to a respective one of the housings 35 by a connector 45 so that the eye-mask 15 can be removably coupled to each of the housings 35 to provide the tension onto the eye-mask 15 as well as to provide functional integration with operations provided by the headphones 10. Accordingly, in some embodiments according to the invention shown in FIG. 3C, the eye-mask 15 includes a connector rather than the loops 25.

In some embodiments according to the invention as illustrated for example in FIG. 3C, functions supported by the eye-mask 15 can be controlled by the headphones 10 via communication through the connector 45 which may include electrical as well as mechanical components. In some embodiments according to the invention, the mechanical integration may be provided by, for example, the connector 45 or loops 25 whereas the functional integration may be via a wireless connection.

In some embodiments, the eye-mask 15 may be integrated with what is sometimes referred to as “ear-buds” instead of the headphones shown in FIGS. 3A-3C. In such embodiments, the “ear-buds” may be functionally integrated with the eye-mask 15.

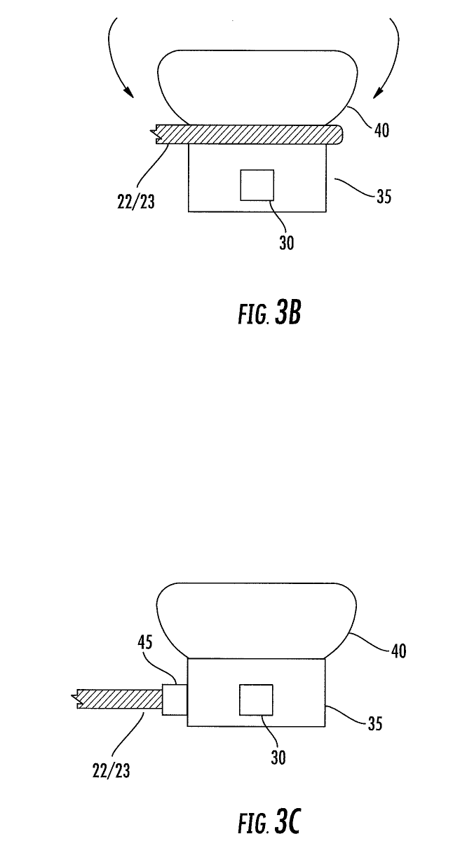

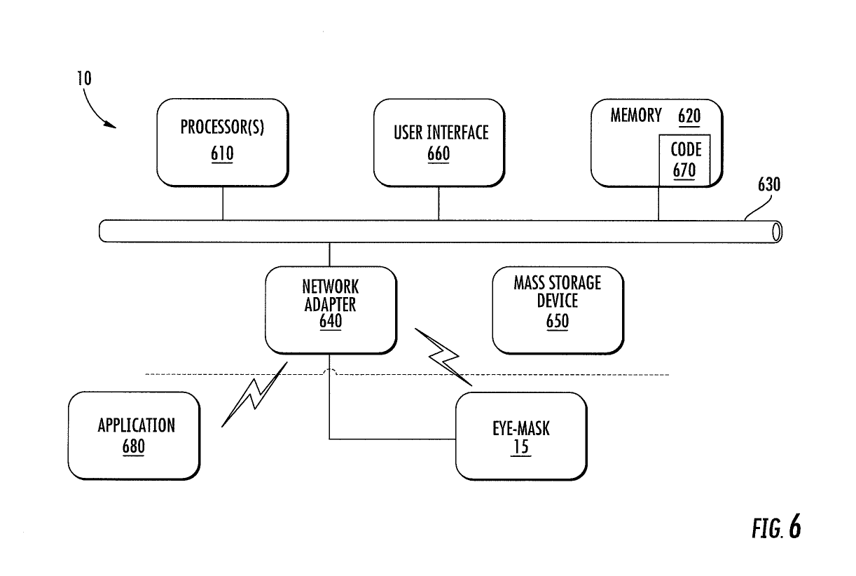

FIG. 4 is a schematic representation of a front exterior surface 50 of the eye-mask 15. It will be understood that the depiction shown in FIG. 4 is not visible to the wearer when the eye-mask 15 is in place during use. As further shown in FIG. 4, the eye-mask 15 is configured to support functional operations such as a signal light 55 which may be used to signal a flight attendant for assistance, or may indicate that the wearer should not be disturbed. For example, in some embodiments, the wearer may manually activate the signal light 55 using an application that runs on the headphones 10 or on a personal electronic device that hosts an application (see FIG. 6 for example showing an application 680 running remotely from the headphones 10 and operatively coupled to the eye-mask 15 via the headphones 10).

As further shown in FIG. 4, the front exterior surface 50 also includes a display 60 which can be used to display images, graphics, and/or video to outside observers. For example, in some embodiments according to the invention, the display 60 can be used to provide a DND (Don Not Disturb) function or other messages to outside observers. In some embodiments, the images, graphics, and/or video can be generated by an application running on the headphones 15 (which may be selected by the wearer or may be selected by the application based on, for example, music being played through the headphones 10). In still further embodiments, the images, graphics, and/or video can include advertising selected by the application based on a destination of the wearer determined by, for example, an itinerary, a message sent by the application, a local message (sent, for example, from inside a vehicle in which the wearer is travelling, or the like).

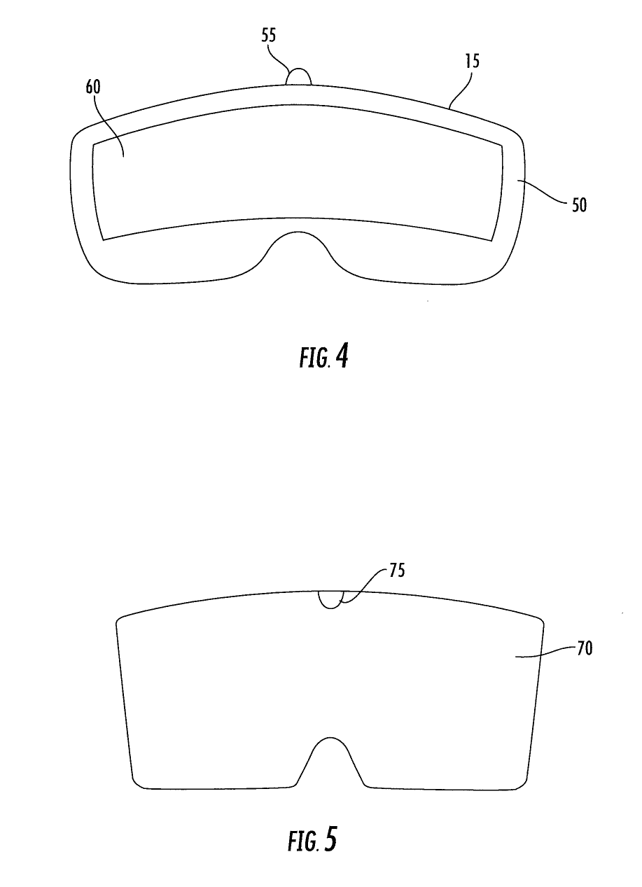

FIG. 5 is a perspective view of an interior surface 70 of the eye-mask 15 in some embodiments according to the invention. Accordingly, the interior surface 70 is visible to the wearer of the eye-mask 15 when in use. As further shown in FIG. 5, the interior surface 70 of the eye-mask 15 can include a wake-up signal 75 (such as a light) which may be used to wake the wearer gradually or suddenly by remote signal. For example, a system associated with a vehicle in which the wearer is travelling may transmit the wake-up signal (directly to the headphones 10 or to an application that is functionally integrated with the headphones 10) based on a time, proximity to a location, or in response to manual input. In still further embodiments according to the invention, the signal light 75 can be used to gradually introduce changing light intensities/color to the wearer under control of the headphones 10 or other application, or system as described above.

FIG. 6 illustrates a block diagram showing an example architecture of the headphones 10, as described herein, and which may implement the operations described above. The headphones 10 includes one or more processors 610 and memory 620 coupled to an interconnect 630. The interconnect 630 may be an abstraction that represents any one or more separate physical buses, point to point connections, or both connected by appropriate bridges, adapters, or controllers. The interconnect 630, therefore, may include, for example, a system bus, a Peripheral Component Interconnect (PCI) bus or PCI-Express bus, a HyperTransport or industry standard architecture (ISA) bus, a small computer system interface (SCSI) bus, a universal serial bus (USB), IIC (12C) bus, or an Institute of Electrical and Electronics Engineers (IEEE) standard 1394 bus, also called “Firewire”.

The processor(s) 610 may be the central processing unit (CPU) of the headphones 10 and, thus, control the overall operation of the headphones 10. In certain embodiments, the processor(s) 610 accomplish this by executing software or firmware stored in memory 620. The processor(s) 610 may be, or may include, one or more programmable general purpose or special-purpose microprocessors, digital signal processors (DSPs), programmable controllers, application specific integrated circuits (ASICs), programmable logic devices (PLDs), field-programmable gate arrays (FPGAs), trusted platform modules (TPMs), or a combination of such or similar devices.

The memory 620 is or includes the main memory of the headphones 10. The memory 620 represents any form of random access memory (RAM), read-only memory (ROM), flash memory, or the like, or a combination of such devices. In use, the memory 620 may contain code 670 containing instructions according to the techniques disclosed herein.

Also connected to the processor(s) 610 through the interconnect 630 are a network adapter 640 and a mass storage device 650. The network adapter 640 provides the headphones 10 with the ability to communicate with remote devices (such as the eye-mask 15) over a network and may be a wireless connection or a wired connection (such as an Ethernet adapter, a Bluetooth adapter, etc.) The network adapter 640 may also provide the headphones 10 with the ability to communicate with other systems such as an external application 680 hosted by a personal electronic device and configured to operate the headphones 10 (such as streaming music to the headphones). The code 670 stored in memory 620 may be implemented as software and/or firmware to program the processor(s) 610 to carry out actions described above. In certain embodiments, such software or firmware may be initially provided to the headphones 10 by downloading it from a remote system through the headphones 10 (e.g., via network adapter 640).

Also connected to the processor(s) 610 through the interconnect 630 is a user interface 660 for the headphones 10. The user interface 660 may receive input from the wearer of the headphone 10, such as via a contact sensitive surface. Though illustrated as a single element, the headphones 10 may include multiple elements as part of the user interface 660. In some embodiments, the user interface 660 may include a plurality of buttons that may be used to activate associated functions of the eye-mask, such as mute, call, wake-up mode, or the like. The user interface 660 may communicate via the interconnect 630 with the memory 620, the processors 610, the network adapter 640 and/or the mass storage device 650 to store, analyze, and/or communicate the input received by the user interface 660 to the headphones 10.

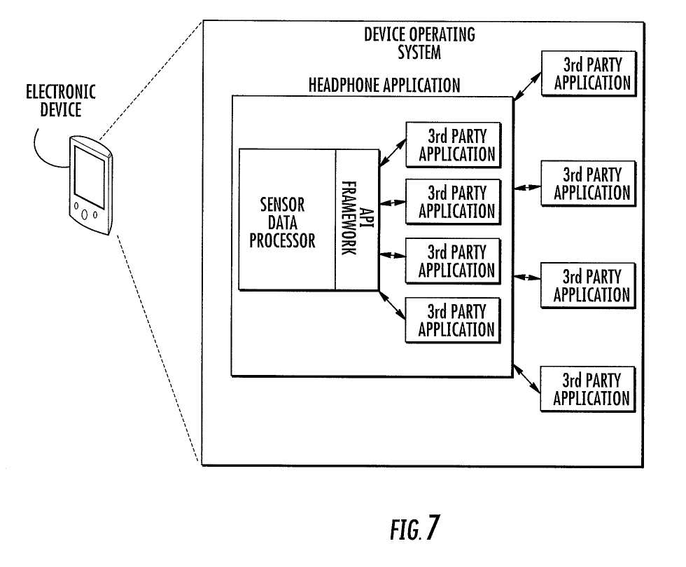

FIG. 7 illustrates an embodiment for a cross-platform application programming interface for the headphones 10 and eye-mask. As illustrated in FIG. 7, an electronic device 30 may run a device operating system. In some embodiments, the device operating system may be the portable electronic device operating system such as iOS or Android.

Within the device operating system, the application 680 may execute. The headphone application 680 may be communicatively coupled to the headphones 10 via the portable electronic device 30. Though illustrated as headphones 10 and application 680 within the figures, it will be understood that the present inventive concepts may apply to any connected wearable device.

Within the operating environment of the application 680, there may be a sensor data processor. The sensor data processor may communicate with sensors on the headphones 10 and/or the connected device 34, such as the eye-mask 15. The sensor data processor may operate to provide data from the sensors to third party applications. For example, the sensor data processor may indicate whether the eye-mask is currently being worn by the user.

As illustrated in FIG. 7, in some embodiments of the present inventive concepts, the integration with the third party applications may be accomplished via an API framework coupled to the sensor data processor. The third party applications may provide respective third party applets which are configured to execute within the application 680. The third party applets may be statically or dynamically linked to the application 680.

The third party applets may be configured to send and/or receive data from the sensor data processor via the API framework. The API framework may be a complete implementation of all the functions by which data may be exchanged between the third party applets and the sensor data processor. Individual ones of the third party applets may implement some or all of the functions defined within the API framework.

Portions of the API framework may support specific classes of devices and/or device implementations. For example, the API framework may define classes such as an AUDIO device and/or a VIDEO device. Third party applets may implement commands to the generic devices and/or may implement customized commands specific to their implementation.

As illustrated in FIG. 7, the third party applets may, in turn, communicate directly to their respective third party applications. The third party applications may also be executing within the device operating system. In some embodiments, the third party applications may communicate with additional externally connected devices.

By integrating with third party applications, the application 680 can provide connective functionality between the headphones 10 and other external devices and/or functions, such as the eye-mask 15.

Embodiments of the present disclosure were described herein with reference to the accompanying drawings. Other embodiments may take many different forms and should not be construed as limited to the embodiments set forth herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the various embodiments described herein. As used herein, the term “and/or” includes any and all combinations of one or more of the associated listed items.

The terminology used herein is for the purpose of describing particular embodiments only and is not intended to be limiting to other embodiments. As used herein, the singular forms “a”, “an” and “the” are intended to include the plural forms as well, unless the context clearly indicates otherwise. It will be further understood that the terms “comprises,” “comprising,” “includes” and/or “including”, “have” and/or “having” when used herein, specify the presence of stated features, integers, steps, operations, elements, and/or components, but do not preclude the presence or addition of one or more other features, integers, steps, operations, elements, components, and/or groups thereof. Elements described as being “to” perform functions, acts and/or operations may be configured to or other structured to do so.

Unless otherwise defined, all terms (including technical and scientific terms) used herein have the same meaning as commonly understood by one of ordinary skill in the art to which various embodiments described herein belong. It will be further understood that terms used herein should be interpreted as having a meaning that is consistent with their meaning in the context of this specification and the relevant art and will not be interpreted in an idealized or overly formal sense unless expressly so defined herein.

As will be appreciated by one of skill in the art, various embodiments described herein may be embodied as a method, data processing system, and/or computer program product. Furthermore, embodiments may take the form of a computer program product on a tangible computer readable storage medium having computer program code embodied in the medium that can be executed by a computer.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wired, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computer environment or offered as a service such as a Software as a Service (SaaS).

Some embodiments are described herein with reference to flowchart illustrations and/or block diagrams of methods, systems and computer program products according to embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create a mechanism for implementing the functions/acts specified in the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall support claims to any such combination or subcombination.

What is claimed:

1. An eye-mask comprising:
an exterior surface configured to face away from a wearer;
an interior surface, opposite the exterior surface, the interior surface configured to contact the wearer and cover eyes of the wearer during use;
a first removable coupling, coupled to a first end of the exterior surface, the first removable coupling configured to removably couple to a first ear piece of a pair of headphones; and
a second removable coupling, coupled to a second end of the exterior surface opposite the first end, the second removable coupling configured to removably couple to a second ear piece of the pair of headphones.

2. The eye-mask of claim 1 wherein the first and second removable couplings comprise first and second elastic loops configured to loop around the first and second ear pieces respectively.

3. The eye-mask of claim 1 wherein the first and second removable couplings comprise first and second connectors configured to connect to first and second ear piece connectors on the first and second ear pieces respectively.

4. The eye-mask of claim 3 further comprising:
a wired or wireless communications interface configured to electrically couple the eye-mask to the pair of headphones.

5. The eye-mask of claim 1 wherein the exterior surface includes a signal light configured to emit light in response to a signal from the pair of headphones.

6. The eye-mask of claim 1 wherein the exterior surface includes a display on a front of the exterior surface, the display configured to display an image.

7. The eye-mask of claim 6 wherein the image is received under control of the pair of headphones.

8. The eye-mask of claim 1 wherein the interior surface includes a light configured to emit light visible to the wearer when in use in response to a wake up signal.

9. The eye-mask of claim 8 wherein the wake up signal is received from the pair of headphones or a separate system external to the pair of headphones.

10. The eye-mask of claim 1 wherein the interior surface includes a display configured to be visible to the wearer when in use.

11. The eye-mask of claim 10 wherein the display is configured to emit a wake up signal configured to be visible to the wearer when in use responsive to a signal from the pair of headphones or a separate system external to the pair of headphones.

12. The eye-mask of claim 11 wherein the signal is based on a time, a proximity to a location, or a manual input.

13. The eye-mask of claim 4 further comprising:
an Application Programming Interface (API) configured to provide control of functions supported by the eye-mask via the wired or wireless communications interface by an application operating on the pair of headphones or a separate system external to the pair of headphones.

14. An eye-mask comprising:
an exterior surface configured to face away from a wearer;
an interior surface, opposite the exterior surface, the interior surface configured to contact the wearer and cover eyes of the wearer during use; and
a first and second removable couplings, coupled to first and second ends of the exterior surface, the first and second removable couplings configured to integrate the eye-mask with a pair of headphones.

15. The eye-mask of claim 14 wherein the first and second removable couplings are configured to mechanically integrate the eye-mask with the pair of headphones.

16. The eye-mask of claim 15 wherein the first and second removable couplings are configured to functionally integrate the eye-mask with the pair of headphones.

17. The eye-mask of claim 14 wherein the exterior surface includes a signal light configured to emit light in response to a signal from the pair of headphones.

18. The eye-mask of claim 14 wherein the exterior surface includes a display on a front of the exterior surface, the display configured to display an image.

19. The eye-mask of claim 18 wherein the image is received under control of the pair of headphones.

20. The eye-mask of claim 14 wherein the first and second removable couplings are separate form one another.

21. An eye-mask comprising:
an exterior surface configured to face away from a wearer;
an interior surface, opposite the exterior surface, the interior surface configured to contact the wearer and cover eyes of the wearer during use;
a first removable coupling, coupled to a first end of the exterior surface, the first removable coupling comprising a first electrical connector configured to removably couple to a first ear piece electrical connector of a pair of headphones; and
a second removable coupling, coupled to a second end of the exterior surface, the second removable coupling comprising a connector configured to removably couple to a second ear piece connector of the pair of headphones.

22. The eye-mask of claim 21 wherein connector comprises a second electrical connector configured to removably couple to a second ear piece electrical connector of the pair of headphones.
